(19) United States
(12) Patent Application Publication
(10) Pub. No.: US 2023/0022779 A1
(43) Pub. Date: Jan. 26, 2023

(54) DETECTION OF VIABLE MICROORGANISMS IN URINE

(71) Applicant: Children's National Medical Center, Washington, DC (US)

(72) Inventors: Michael Hsieh, Bethesda, MD (US); Olivia Lamanna, Washington, DC (US); Albert Lee, Washington, DC (US); Kenji Ishida, Washington, DC (US)

(21) Appl. No.: 17/870,299

(22) Filed: Jul. 21, 2022

Related U.S. Application Data
(60) Provisional application No. 63/224,700, filed on Jul. 22, 2021.

(51) Int. Cl. C12Q 1/689

(52) U.S. Cl. CPC C12Q 1/689; C12Q 1/6806

(57) ABSTRACT

The present invention provides a method for the detection of viable microorganisms in urine using DNA-crosslinking agent to differentiate between dead and live microorganisms. The DNA-crosslinking agent can penetrate cells which have compromised cell membranes, such as dead cells. The method of the present application comprises amplifying DNA in the urine sample which is pre-treated with the DNA-crosslinking agent. In addition, the method includes the step of centrifugation to remove supernatant from the urine.

### REFERENCE TO RELATED APPLICATION

This application claims the benefit of the filing date under 35 U.S.C. 119(e) of U.S. Provisional Patent Application No. 63/224,700, filed on Jul. 22, 2021, the entire contents of which are incorporated herein by reference.

### FIELD OF THE INVENTION

The present invention pertains to the fields of detecting and/or quantitating metabolically active (e.g., live) microorganisms in urine.

### BACKGROUND OF THE INVENTION

The existence of bacteria in urine, such as the urine microbiome, has recently been reported which refutes the previous belief that urine is sterile. Several studies have shown an association between the urine microbiome and various urological diseases. However, many of these studies rely on PCR (polymerase chain reaction) to identify specific organisms by amplifying total DNA material.

These methods are not selective in the source of DNA so the indiscriminate amplification of both relic DNA (from dead bacteria) and DNA from viable bacteria can bias results. Clinical tests for urinary tract infection (UTI) that rely on conventional PCR have similar limitations. The identification of microorganisms in urine regarding a urinary tract infection, such as the study of the urine microbiome, is limited by the inability of PCR to differentiate DNA from metabolically active (live) and inactive (dead) bacteria.

### SUMMARY OF THE INVENTION

The present application provides a method for the detection of viable microorganisms in urine using DNA-crosslinking agent, such as propidium monoazide (PMA) dye, to differentiate between dead and live microorganisms. For example, PMA dye can penetrate cells which have compromised cell membranes, such as dead cells. After the penetration, PMA can covalently bind to DNA which renders the DNA unable to be amplified by PCR (polymerase chain reaction).

The present application provides a method of detecting and/or quantitating metabolically active (e.g., live) microorganisms in a urine sample. The method of the present application comprises amplifying DNA in the urine sample which is pre-treated with a DNA-crosslinking agent that preferentially or exclusively penetrates metabolically inactive (e.g., dead) but not metabolically active (e.g., live) microorganisms, wherein the urine sample comprises, consists essentially of, or consists of insoluble components separated from the aqueous portion of urine. The microorganism may comprise bacteria, yeast, virus, or combinations thereof. In the method of the present application, the urine sample can be obtained by re-suspending said insoluble components in a buffer after centrifuging the urine to precipitate insoluble components, wherein the buffer may be one which is compatible for DNA amplification, such as PBS (phosphate buffered saline). The insoluble components can be precipitated from the urine by centrifugation at about 5,000 g or above, for, e.g., at least about 8 minutes.

In certain embodiments, in the method of the present application, the urine is diluted by the buffer (e.g., 1:2 dilution) before centrifugation.

In certain embodiments, the DNA-crosslinking agent is propidium monoazide (PMA), propidium monoazide derivatives such as PMaxx and PMaxx Plus, ethidium monoazide bromide (EMA), a chromium metabolite (thiol reactions), a simple aryl azide, a fluorinated aryl azide such as a nitrene generating reagent, or a benzophenone derivative.

In one embodiment, the DNA-crosslinking agent is PMA.

In certain embodiments, the urine sample is pre-treated with the DNA-crosslinking agent (e.g., 200 μM PMA) by incubating in dark for 15 or more minutes, followed by LED-light-induced crosslinking for 20 or more minutes.

In certain embodiments, the DNA amplification is performed by polymerase chain reaction (PCR), rolling circle amplification (RCA), loop mediated isothermal amplification (LAMP), nucleic acid sequence based amplification (NASBA), strand displacement amplification (SDA), multiple displacement amplification (MDA), ligase chain reaction (LCR), helicase dependant amplification (HDA), or ramification amplification method (RAM).

In one embodiment, DNA amplification is performed by quantitative PCR (qPCR).

In certain embodiments, in the method of the present application, two or more marker genes from the bacteria (e.g., comprising the uidA gene of E coli.) are simultaneously amplified using PCR.

In certain embodiments, an antibiotic resistance gene from the bacteria, and/or a bacterial species-specific gene are amplified using PCR.

In certain embodiments, the bacteria comprise E. coli, Pseudomonas spp. (such as Pseudomonas aeruginosa), Klebsiella spp. (such as Klebsiella pneumoniae), Proteus spp. (such as Proteus mirabilis), Enterococcus spp. (such as Enterococcus faecalis), Enterobacter, Coagulase-negative Staphylococci, Staphylococcus aureus, and/or Acinobacter.

In certain embodiments, the microorganism comprises Acinetobacter baumannii, Actinotignum schaalii, Aerococcus urinae, Alloscardovia omnicolens, Candida albicans, Candida auris, Candida glabrata, Candida parapsilosis, Citrobacter freundii, Citrobacter koseri, Corynebacterium riegelii, Enterococcus faecalis, Enterococcus faecium, Escherichia coli, Klebsiella oxytoca, Coagulase-neg. staphylococci, Viridans group streptococci, Enterobacter group, BK virus, HHV-5 (CMV), HHV-6, HHV-1, HHV-2 (HSV 1/2), JC virus, Klebsiella pneumoniae, Morganella morganii, Mycobacterium tuberculosis, Mycoplasma hominis, Pantoea agglomerans, Proteus mirabilis, Providencia stuartii, Pseudomonas aeruginosa, Serratia marcescens, Staphylococcus aureus, Streptococcus agalactiae, Ureaplasma urealyticum, Chlamydia trachomatis, Neisseria gonorrhoeae, Trichomonas vaginalis, or combinations thereof.

In certain embodiments, said urine is from a mammalian subject (e.g., human or mouse).

In certain embodiments, the human has, suspected to have, is predisposed to or is at high risk of having UTI (uninary tract infection).

In certain embodiments, the UTI is cystitis, pyelonephritis, or complicated UTI (cUTI). The mammalian subject is a human having urinary catheterization (e.g., human having chronic catheter) or have had urinary catheterization (e.g., human having intermittent catheterization).

In certain embodiments, the mammalian subject is a human undergoing or having completed treatment (e.g., antibiotic treatment) for UTI.

In certain embodiments, the mammalian subject is a human at increased risk for recurrent UTI, such as human with intermittent catheterization, Spina bifida, lower urinary tract dysfunction, diabete, and/or immunosuppression.

In certain embodiments, the mammalian subject is a female human, such as a woman who is pregnant, or a woman who has given recent (e.g., within 1, 2, 3 day, 1, 2, 3, weeks, 1, 2, 3 months) vaginal birth.

In certain embodiments, the mammalian subject is a geriatrics patient.

It should be understood that any one embodiment described herein, including embodiments that have only been described in the examples or claims, can be combined with one or more any other embodiments, unless such combination is disclaimed or improper.

### BRIEF DESCRIPTION OF THE DRAWING FIGURES

Further features of the inventive concept, its nature and various advantages will be more apparent from the following detailed description, taken in conjunction with the accompanying figures:

FIG. 1A shows CT values of PMA treated and untreated samples of dead bacteria resuspended in mouse urine or PBS according to an exemplary embodiment.

FIG. 1B shows the difference in CT (dCT) values of dead bacteria resuspended in various dilutions of urine according to an exemplary embodiment.

FIG. 1C shows CT values of dead bacteria treated with PMA in the presence of human and mouse urea levels according to an exemplary embodiment.

FIG. 1D shows CT values of dead bacteria in urine with or without resuspending the contents in PBS before PMA treatment according to an exemplary embodiment.

FIG. 2A shows fraction dead in the background of live bacteria according to an exemplary embodiment.

FIG. 2B shows fraction live in the background of dead bacteria according to an exemplary embodiment.

FIG. 2C shows the plots of CFUs per ml against the respective CT values according to an exemplary embodiment.

FIG. 3 shows an in vivo experimental plan according to an exemplary embodiment.

FIG. 4 shows a flow chart of the method of the present application according to an exemplary embodiment.

FIG. 5 shows that PMA-qPCR can be used to determine whether urine bacteria are susceptible to antibiotic treatment.

### DETAILED DESCRIPTION OF THE INVENTION

Throughout this description, the preferred embodiments and examples provided herein should be considered as exemplar, rather than as limitations of the present application.

The present application provides a method to differentiate metabolically active (e.g., live) from inactive (e.g., dead) bacteria in urine using a DNA-crosslinking agent, such as propidium monoazide (PMA) dye. The DNA-crosslinking agent can penetrate cells which have compromised cell membranes, such as dead cells. After the penetration, the DNA-crosslinking agent can covalently bind to DNA which renders the DNA unable to be amplified by, e.g., PCR (polymerase chain reaction). The present application provides a method of detecting and/or quantitating metabolically active (e.g., live) microorganisms in a urine sample.

In one embodiment, the method includes the step of centrifugation to remove supernatant from the urine. After decanting the urine supernatant, the pellet is resuspended in a buffer, preferably a buffer either comparible with or otherwise does not interfere with subsequent sample DNA amplification, such as PBS, prior to using the DNA-crosslinking agent. The method of the present application is effective for increasing the efficiency of the DNA-cros slinking agent by removing highly concentrated urine filtrate and relic DNA (DNA from dead bacteria) to reduce interference. In addition, the process is effective in retaining microorganisms (viable or nonviable), such as bacteria, which are present in the urine. The method of the present application comprises amplifying DNA in the urine sample which is pre-treated with a DNA-crosslinking agent that preferentially or exclusively penetrates metabolically inactive (e.g., dead) but not metabolically active (e.g., live) microorganisms, wherein the urine sample comprises insoluble components separated from the aqueous portion of urine. The microorganism to be detected comprises bacteria, yeast, virus, or combinations thereof. In the method of the present application, the urine sample can be obtained by re-suspending said insoluble components in a buffer after centrifuging the urine to precipitate the insoluble components. The insoluble components can be precipitated from the urine by centrifugation.

In one embodiment, the insoluble components are precipitated from the urine by centrifugation, at a speed sufficiently high to precipitate insoluble components of the urine, such as at about 3,000 g, about 4,000 g, about 5,000 g, about 6,000 g, about 7,000 g, about 8,000 g or above, for at least about 3 minutes, about 4 minutes, about 5 minutes, about 7 minutes, about 8 minutes, about 9 minutes, about 10 minutes, or more than 10 minutes.

In one embodiment, the process of the present application includes the step of using the DNA-crosslinking agent, such as PMA, at a final concentration of 200 μM. In addition, the steps of dark and light incubations can be extended. Furthermore, there is no need for the final resuspension in PBS after PMA treatment comparing to conventional method. The sample can go straight to the DNA extraction workflow and subsequently for performing PCR, such as qPCR. Performing these specific steps enables obtaining dCT values which are comparable in urine.

Overall, the method of the present application provides a sensitive method to identify metabolically active, or live microorganisms, such as bacteria, in urine, by utilizing DNA-crosslinking agent, such as PMA, allowing PCR-based studies.

The study of the urine microbiome and the identification of organisms present during a urinary tract infection utilizing PCR are limited by the inability of PCR to differentiate DNA material from metabolically active (live) and inactive (dead) bacteria. PMA dye can penetrate cells with compromised cell membranes and covalently binds to DNA rendering it unable to be amplified by PCR. Due to the high sensitivity of the method, the method of the present application is an invaluable asset to clinical diagnosis.

In one embodiment, the urine is diluted by the buffer (e.g., 1:2 dilution) before centrifugation. The DNA-crosslinking agent is propidium monoazide (PMA), propidium monoazide derivatives such as PMaxx and PMaxx Plus, ethidium monoazide bromide (EMA), a chromium metabolite (thiol reactions), a simple aryl azide, a fluorinated aryl azide such as a nitrene generating reagent, or a benzophenone derivative. In a preferred embodiment, the DNA-crosslinking agent is PMA. In one aspect, the urine sample is pre-treated with the DNA-crosslinking agent (e.g., 200 μM PMA) by incubating in dark for, 5 minutes, 10 minutes, 15 minutes, 20 minutes, 5-20 minutes, 10-20 minutes, 10-15 minutes or longer, followed by LED-light-induced crosslinking for 10 minutes, 15 minutes, 20 minutes, 25 minutes, 30 minutes, 5-25 minutes, 10-25 minutes, 15-30 minutes or longer. The DNA amplification can be performed by polymerase chain reaction (PCR), rolling circle amplification (RCA), loop mediated isothermal amplification (LAMP), nucleic acid sequence based amplification (NASBA), strand displacement amplification (SDA), multiple displacement amplification (MDA), ligase chain reaction (LCR), helicase dependant amplification (HDA), or ramification amplification method (RAM). In a preferred embodiment, DNA amplification is performed by quantitative PCR (qPCR).

The method of the present application can be used as an additional tool for the diagnosis of UTI. It can be coupled with current PCR UTI diagnostic technology to ensure positive PCR results are derived from metabolically active bacteria only. The method of the present application can also be used to access successful antibiotic treatment of UTI especially in those with recurrent UTI. It can be used to monitoring the infection to ensure that no residual viable bacteria is present after medical treatment. When the method is used in urine microbiome studies, it can improve the identification of only live urine microbiome using PCR. This method could be used to measure antibiotic resistance genes coming from live bacteria, and thus guide antibiotic therapy, such as combining with bacterial species-specific viability PCR. Viability PCR may facilitate more rapid UTI diagnosis than waiting for urine culture results, which can take 1-3 days. Viability PCR can be used to measure virulence genes coming from live bacteria, and thus guide therapy, such as combining with bacterial species-specific viability PCR. The method can also be developed as a multiplex format to detect typical uropathogens in a single PCR reaction, saving on reagent and supply costs.

Thus in a related aspect, the invention provides a method for diagnosing and/or treatment of a urinary track infection (UTI), such as complicated UTI (cUTI) or uncomplicated UTI (uUTI), including cystitis and pyelonephritis, the method comprising determining the presence and/or amount of a live bacteria commonly found or associated with the UTI in a urine sample obtained from a subject having UTI, at risk of having UTI, or suspected of having UTI, using any of the method of the invention. In certain embodiments, the method further comprising treating the subject confirmed to have said live bacteria with a therapeutically effective amount of a medicament effective to treat UTI, such as an antibiotic. Suitable antibiotic include one or more of: nitrofurantoin, trimethoprim/sulfamethoxazole, methenamine, phenazopyridine, fosfomycin, cephalosporin, amoxicillin/clavulanic acid, fluoroquinolone, ciprofloxacin, or tetracycli class compounds including tigecycline, eravacycline and omadacycline.

In certain embodiments, the method further comprises determining the presence and/or amount of the live bacteria in a follow-up urine sample following treatment, in order to determine the efficacy of the treatment to inhibit infection by the bacteria.

In a further related apsect, the invention provides a method to verify a positive UTI diagnosis based on a different methodology, the method comprising determining the presence and/or amount of a live bacteria detected to be positive by the different methodology in a urine sample obtained from a subject having UTI, at risk of having UTI, or suspected of having UTI, using any of the method of the invention, wherein a positive result is indicative that the different methodology is accurate in UTI diagnosis.

In a further related aspect, the invention provides a method to assess the effectiveness of a treatment regimen for UTI, in a subject having or suspected of having recurrent UTI after the treatment, the method comprising determining the presence and/or amount of a live bacteria commonly found or associated with the UTI (and/or a live bacteria previously found in the subject's urine) in a urine sample obtained from the subject, using any of the method of the invention, wherein identification of the live bacteria in the urine sample is indicative that UTI has recurred or replased following the treatment. In certain embodiments, the method further comprises comparing the level/amount of the live bacteria in the sample to that of a previous urine sample prior to said treatment, in order to determine whether the treatment is effective to inhibit the growth of the live bacteria.In certain embodiments, the method further comprises determining the absence/presence of an antibiotic gene in the live bacteria, in order to facilitate the selection of an antibiotic not likely to be resistant by the bacteria for further treatment.

In certain embodiments, said urine is from a mammalian subject (e.g., human or mouse).

In certain embodiments, the human has, suspected to have, is predisposed to or is at high risk of having UTI (uninary tract infection).

In certain embodiments, the UTI is cystitis, pyelonephritis, or complicated UTI (cUTI).

In certain embodiments, the mammalian subject is a human having urinary catheterization (e.g., human having chronic catheter) or have had urinary catheterization (e.g., human having intermittent catheterization).

In certain embodiments, the mammalian subject is a human undergoing or having completed treatment (e.g., antibiotic treatment) for UTI.

In certain embodiments, the mammalian subject is a human at increased risk for recurrent UTI, such as human with intermittent catheterization, Spina bifida, lower urinary tract dysfunction, diabete, and/or immunosuppression.

In certain embodiments, the mammalian subject is a female human, such as a woman who is pregnant, or a woman who has given recent (e.g., within 1, 2, 3 day, 1, 2, 3, weeks, 1, 2, 3 months) vaginal birth.

In certain embodiments, the mammalian subject is a geriatrics patient.

In the method of the present application, two or more marker genes from the bacteria (e.g., comprising the uidA gene of E coli.) can be simultaneously amplified using PCR.

In certain embodiments, an antibiotic resistance gene from the bacteria, and/or a bacterial species-specific gene are amplified using PCR.

In certain embodiments, the bacteria may comprise E. coli, Pseudomonas spp. (such as Pseudomonas aeruginosa), Klebsiella spp. (such as Klebsiella pneumoniae), Proteus spp. (such as Proteus mirabilis), Enterococcus spp. (such as Enterococcus faecalis), Enterobacter, Coagulase-negative Staphylococci, Staphylococcus aureus, and/or Acinobacter.

In certain embodiments, the microorganism comprises Acinetobacter baumannii, Actinotignum schaalii, Aerococcus urinae, Alloscardovia omnicolens, Candida albicans, Candida auris, Candida glabrata, Candida parapsilosis, Citrobacter freundii, Citrobacter koseri, Corynebacterium riegelii, Enterococcus faecalis, Enterococcus faecium, Escherichia coli, Klebsiella oxytoca, Coagulase-neg. staphylococci, Viridans group streptococci, Enterobacter group, BK virus, HHV-5 (CMV), HHV-6, HHV-1, HHV-2 (HSV 1/2), JC virus, Klebsiella pneumoniae, Morganella morganii, Mycobacterium tuberculosis, Mycoplasma hominis, Pantoea agglomerans, Proteus mirabilis, Providencia stuartii, Pseudomonas aeruginosa, Serratia marcescens, Staphylococcus aureus, Streptococcus agalactiae, Ureaplasma urealyticum, Chlamydia trachomatis, Neisseria gonorrhoeae, Trichomonas vaginalis, or combinations thereof.

The experimental results indicate that the PMA-based urine PCR method of the present application is a sensitive method to distinguish viable and non-viable bacteria in the urine. The method of the present application can effectively remove urine prior to PMA treatment to avoid the inhibitory effect.

Interestingly, urea was examined and was determined to not impact the effectiveness of PMA. Urea, a by-product of amino acid metabolism, is one of the most abundant solutes found in urine. Mouse urine has a high urea concentration compared to humans so it is reasonable to speculate urea could be influencing PMA function in mice. The finding of similar dCT values of spiked-urea solution and PBS suggests that the presence of urea in urine does not inhibit PMA's effectiveness. The inhibitory effect of urine is likely due to a matrix effect.

The method of the present application allows differentiating metabolically active from inactive bacteria in urine. After centrifuging urine to remove the supernatant and resuspending the pellet in PBS, dCT values similar to those in PBS and those reported in the literature were obtained. This demonstrates an appropriate method to utilize PMA in urine and thus allows PCR-based studies to only identify metabolically active bacteria in urine. The ability to differentiate bacteria's metabolic states is crucial as there is much clinical applicability. Specifically, the method allows differentiating relic DNA and live DNA when studying urine microbiome. Additionally, it allows solely identification of metabolically active bacteria in urine in those exhibiting UTI symptoms when using PCR to identify the microorganisms.

In an embodiment, non-culturable but still viable E. coli were identified after completion of antibiotic treatment. Non-culturable but viable bacteria have been found and shown in many previous studies. However, it has never been shown in post antibiotic treated urine. By using PMA combined with a negative culture in the non-selective LB agar, only live bacteria were isolated to show its existence after antibiotic treatment. This may explain why despite treatment with susceptibility guided antibiotic treatment and a negative test of cure, some patients have recurrence of urinary tract infection with the same pathogen and susceptibility. No growth on the non-selective LB agar further confirmed how these bacteria are not culturable.

### EXAMPLES

The features and properties of the methods of the present application are shown in the examples which illustrate the benefits and advantages of the present invention.

### Example 1. Experimental Methods

Bacteria Preparation: Uropathogenic Escherichia coli strain, UTI89, from glycerol stock was used to inoculate a LB agar plate (Sigma-Aldrich, St. Louis, Mo.). The plate was incubated for 24 hours at 37° C. A single colony was picked and transferred to 10 ml LB broth in a 125 ml Erlenmeyer flask. The culture was incubated overnight at 37° C. without shaking. Twenty-five microliters of the 10 ml culture were transferred to 25 ml of LB broth in a 250 ml Erlenmeyer flask. The culture was incubated overnight at 37° C. without shaking. The culture was centrifuged at 5000×g for 5 minutes at 4° C. The supernatant was decanted and the bacteria pellet was resuspended in 10 ml of sterile phosphate buffered saline (PBS) (Thermofisher Scientific, Waltham, Mass.). One ml of this suspension was added to 9 ml sterile PBS to create a 1:10 dilution. The optical density (OD) 600nm value was analyzed using the NanoDrop-1000 (Thermofisher Scientific, Waltham, Mass.) to be about 0.50 which corresponds to 1-2×107 colony-forming units (CFU) per 50 ul.

Mouse Urine Collection: A mouse was placed above sterile parafilm and gently pressed or tapped on the lower back. Urine was aspirated from the sterile parafilm. New parafilm was used for each mouse. Urine was then placed on ice and immediately processed.

Development of standard curve: Mixing of live and dead bacteria with urine: Defined quantities of isopropanol-killed bacteria, live bacteria, or PBS were mixed to a total volume of 100 ul. With an unchanging amount of live bacteria solution, dead bacteria were added to achieve 1:10, 1:100 and 1:1000 dead to live bacteria dilutions. In a similar manner, live bacteria were added to an unchanging amount of dead bacteria to generate a standard curve. Undiluted live and dead cultures were also used. Urine was spiked with 50 ul of these bacteria solutions. The mixture was then centrifuged at 5000 g, resuspended with 100 uL of PBS, treated with PMA, and the DNA was extracted.

### Example 2. Study the Effect of Urine on PMA

Bacteria killing methods: 500 uL of E. coli with OD value of about 0.5 was mixed with isopropanol (Sigma-Aldrich, St. Louis, Mo.) to achieve a final concentration of 70%. After 10 minutes, the mixture was centrifuged at 8000×g for 10 min. The supernatant was removed and the pellet was resuspended in 100 ul of PBS. 100 ul of the dead bacteria was plated on LB agar plates and incubated at 37° C. overnight to confirm the successful killing of the bacteria.

Spiking of urine: Mouse urine was serially diluted to a ratio of 1:2, 1:4, 1:8, and 1:12 with PBS. Subsequently, 50 uL of all live or all dead bacteria was added to 50 ul of the various dilutions of urine, or undiluted urine. The samples were then either treated with PMA or left untreated.

PMA treatment: Under minimal light, PMAxx Dye (referred to as PMA) (Biotium, Fremont, Calif.) with a concentration of 20 mM was diluted with nuclease-free water (Sigma-Aldrich, St. Louis, Mo.) to a final concentration of 10 mM. It is then added to the bacteria mixture in a 1:100 ratio. After which the samples were incubated for 15 mins in the dark with gentle agitation. The samples were placed in a LED lightbox (Biotium, Fremont, Calif.) for 20 minutes to induce PMA cros slinking of DNA. The supernatant was removed and the pellet was reconstituted with PBS to its original volume.

DNA extraction and quantification: DNA was isolated using the DNeasy PowerSoil Pro Kit (Qiagen, Germantown, Md.) according to kit instructions. However, DNA was eluted from the column with 25 ul of nuclease-free water. PCR was performed targeting the E. coli uidA gene with TaqMan (Invitrogen, Waltham, Mass.).

### Example 3. Study the Effect of Urea on PMA

Urea (Sigma-Aldrich, St. Louis, Mo.) was diluted with PBS to two different concentrations; 285 mM, corresponding to urea level in humans and 1800 mM, corresponding to urea level in mice. Fifty uL of all live or all dead bacteria was added to 50 uL of the urea solutions. DNA of PMA treated and untreated samples were extracted, amplified and compared.

### Example 4. In Vitro Mouse Work

Inoculation via transurethral catheterization of mice: All animals were allotted a 7 days acquisition period after arrival to the animal facility. 24 week old female C3H/HeOuJ mice (stock no: 000635, The Jackson Laboratory, Bar Harbor, Me.) were used in this study.

Catheterization was completed one mouse at a time. The mouse was anesthetized using 2% laminar flow of isoflurane in a chamber and then moved to a nose cone where it was placed in the supine position. The paws were gently squeezed to confirm full anesthetization. Any urine in the bladder was expressed by gently pressing on the lower abdomen. A 24 g×¾ inch angiocath (Clint Pharmaceuticals, Old Hickory, Tenn.) was attached to the prepared 1 ml syringe containing the inoculant. The angiocath was lubricated (DynaLub Sterile Lubricating Jelly, Amazon, Seattle, Wash.) and transurethrally inserted into the bladder. 100u1 of the respective inoculant was instilled slowly into the bladder and the angiocath remained inserted for 30 seconds to prevent leakage of the inoculant. The angiocath was slowly retracted and the mouse was returned to its cage.

Antibiotic treatment: Ciprofloxacin (Sigma-Aldrich, St. Louis, Mo.) was diluted to achieve a concentration of 2 mg/ml. Five days after inoculation mice were treated with intraperitoneal 10 mg/kg ciprofloxacin injection twice a day. This regimen was selected as it mimics a plasma peak level of 500 mg oral administration in humans and had been shown previously to adequately treat UTI in mice.

Urine collection: Mouse urine was collected on ice the day after completion of antibiotic treatment by using methods outlined above. Each urine sample was pooled from the same group of mice (3-4 mice/group). The urine was either serially diluted and plated in triplicate on LB agar or prepared for PMA treatment. Fifty ul of PBS was added to the urine and the solution was centrifuged and treated with PMA as outlined above. DNA was extracted and the E. coli uidA gene was amplified by TaqMan PCR according to previously described methods.

### Example 5. PMA Efficacy

Known amounts of viable or non-viable uropathogenic E. coli (UTI89), or PBS control were mixed with mouse urine. The samples remained in urine or were centrifuged and resuspended. In the dark, 100 uM PMA dye was incubated for 15 min, then the samples were incubated in a blue LED lightbox for 20 minutes to induce PMA crosslinking of DNA. The DNA was isolated using the PowerSoil Pro kit and taqman PCR was performed targeting the E. coli uidA gene. Mice were inoculated with 1×10⁸ E. coli (UTI89). Five days post-inoculation, mice subsequently were treated with ciprofloxacin for 3 days. One day after the completion of ciprofloxacin treatment, an aliquot of urine was plated on non-selective LB agar and another aliquot was treated with PMA.

PMA's efficiency in eliminating non-viable DNA signals significantly decreases in urine (dCT=1.58) when compared to in PBS (dCT=13.69). This discrepancy diminishes after resuspending the urine supernatant in PBS prior to PMA treatment. In 3 of 5 groups of mice that were given a UTI, no bacteria had grown on the non-selective LB agar; however, there was PCR amplification of E. coli after PMA treatment in 2 of those 3 samples. This persistent bacteria signal after antibiotic treatment may illustrate the existence of viable, but nonculturable E. coli. Furthermore, a PMA-based urine PCR is an appropriate method to distinguish viable and non-viable bacteria in urine.

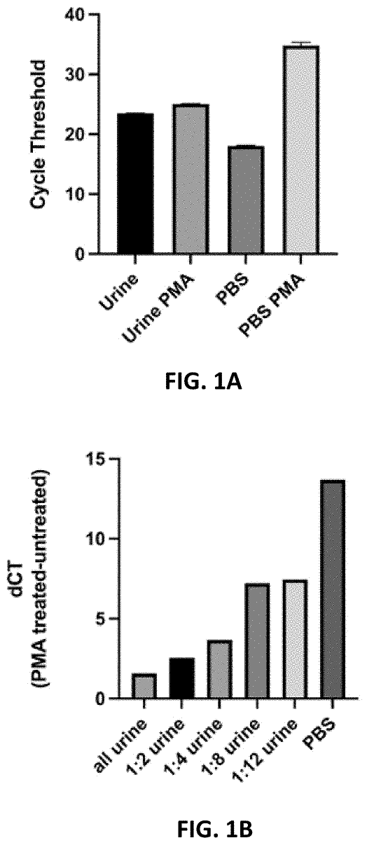

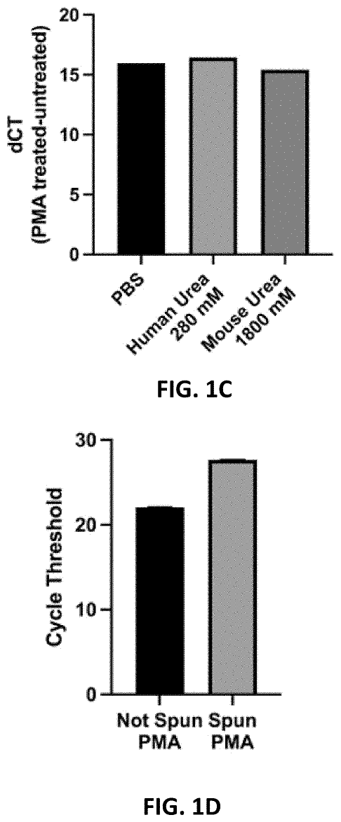

The experimental data shows that urine interferes with PMA efficacy. For PMA to be effective in urine, the DNA sample in the urine must be spun down after its collection and then resuspended in PBS before cross-linking treatment with DNA cross-linking agents such as PMA. FIG. 1A shows CT (PCR Cycle Threshold) values of PMA treated and untreated samples of dead bacteria resuspended in mouse urine or PBS. FIG. 1B shows the difference in CT (dCT) values of dead bacteria resuspended in various dilutions of urine. FIG. 1C shows CT values of dead bacteria treated with PMA in the presence of human and mouse urea levels. FIG. 1D shows CT values of dead bacteria in urine with or without resuspending the contents in PBS before PMA treatment. These are representative data and all experiments were repeated three times.

The experimental data shows that urine interferes with PMA efficacy. Delta CT (dCT) between PMA treated and non-PMA treated all dead samples in the urine (dCT 1.58) was about one tenth of that of those in PBS (dCT 13.69). Additionally, dCTs improved as the urine became more diluted as shown in FIG. 1B. However, the increase in dCT plateaued at a dilution of 1:8. These findings indicated that urine inhibits PMA activity, and the interference can be removed by separating sample DNA from the other components in the urine.

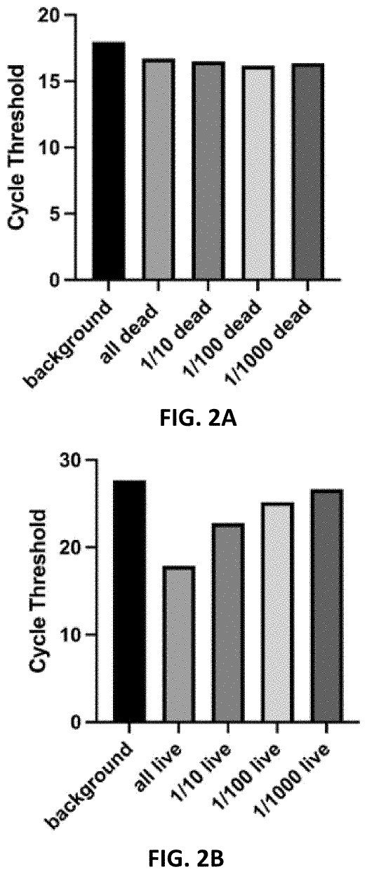

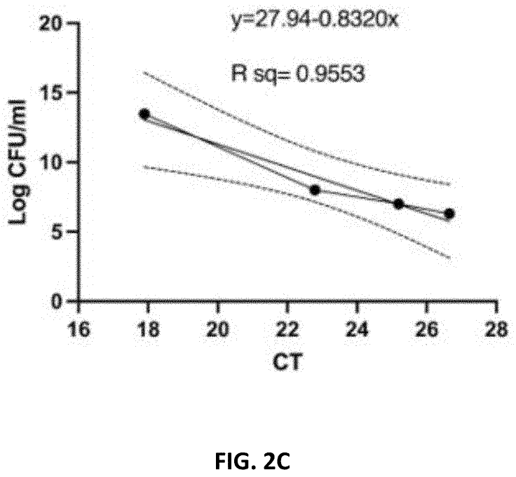

In addition, the experimental data shows that urea does not affect PMA efficacy. PMA is most efficient when most of the bacteria is non-viable. All samples were prepared accordingly, spun and resuspended in PBS before PMA treatment. FIG. 2A shows fraction dead in the background of live bacteria according to an exemplary embodiment. FIG. 2B shows fraction live in the background of dead bacteria according to an exemplary embodiment. FIG. 2A shows that CT values of samples with an unchanging amount of live bacteria and various amounts of dead bacteria. FIG. 2B shows that CT values of samples with an unchanging amount of dead bacteria and various amounts of live bacteria. FIG. 2C shows that before PMA treatment, an aliquot of the samples were serial diluted, plated on LB agar plates, and colonies forming units (CFU) were counted after 24 hours. CFUs per ml were plotted against the respective CT values. The linear regression and 95% confidence interval band is shown. All experiments were repeated 3 times.

The experimental data shows that urine interferes with PMA efficacy. The two urea levels, one similar to human urine and the other similar to mice urine, produced a dCT between PMA treated and untreated all dead samples was similar to that of PBS (dCT PBS 15.98, urea-human 16.42, urea-mice—15.43) as shown in FIG. 2C. Illustrating urea is not the substance in urine that is inhibiting PMA.

The results show that centrifugation of urine and reconstitution with PBS restores PMA efficacy in urine. Removal of urine by centrifugation and subsequent resuspension of the pellet in PBS allows us to differentiate the amount of live vs dead bacteria. However, PMA and the downstream PCR is more effective in detecting the different states of bacteria when there is mostly dead bacteria present in solution. Varying amounts of non-viable bacteria have similar average CT values to the viable control (CT=16.35), suggesting the amount of non-viable bacteria does not influence the detection of viable bacteria as shown in FIG. 2A and FIG. 2B. Similarly, the CT values of samples with an equivalent amount of non-viable bacteria and varying amounts of viable bacteria has a strong negative correlation (R2=0.9553) with measured colony forming units (CFU).

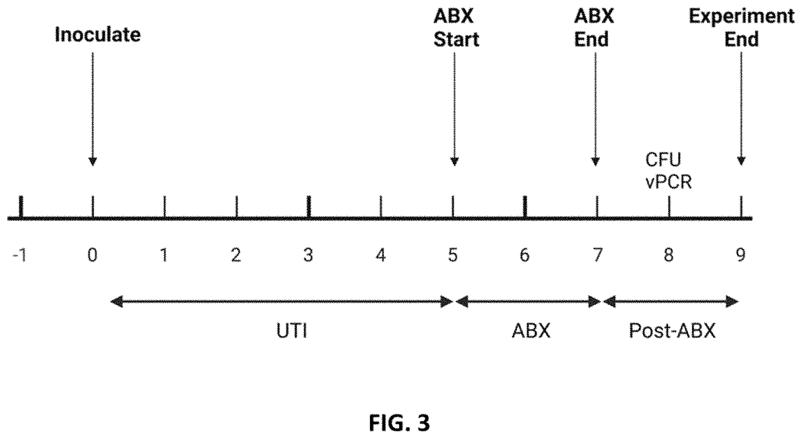

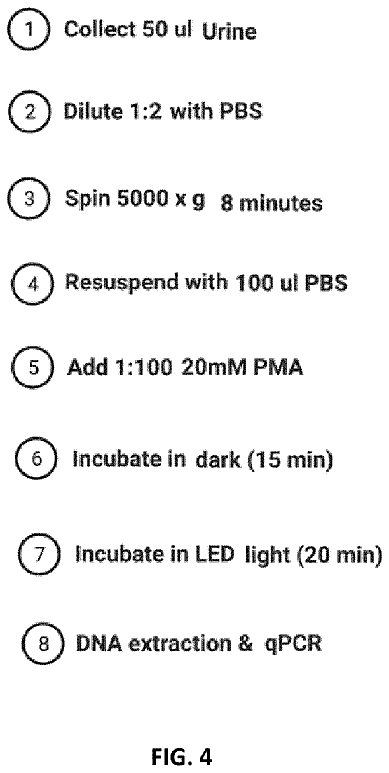

The results indicate the detection of non-culturable but viable bacteria in urine after antibiotic treatment in mice. One day after the completion of antibiotic treatment, 3 out of the 5 groups showed no growth on the non-selective LB agar. However, after PMA treatment, rendering all dead bacterial DNA undetectable, PCR still showed amplification, indicating the presence of live bacteria. Out of those 3 groups, 2 groups had 2.5×10⁴ and 6×10⁵ CFU/ml E. coli calculated based on the generated standard curve as shown in FIG. 2C. FIG. 3 shows an in vivo experimental plan of the present application according to an exemplary embodiment. FIG. 4 shows a flow chart of the method of the present application according to an exemplary embodiment.

It is to be understood that the present invention is not to be limited to the exact description and embodiments as illustrated and described herein. To those of ordinary skill in the art, one or more variations and modifications will be understood to be contemplated from the present disclosure. Accordingly, all expedient modifications readily attainable by one of ordinary skill in the art from the disclosure set forth herein, or by routine experimentation therefrom, are deemed to be within the true spirit and scope of the invention as defined by the appended claims.

### Example 6. Study of PMA in the Context of Bacteria Treated with Antibiotics

A bacteria reference strain for Klebsiella pneumoniae (ATCC 13883), prepared in a similar manner described in Example 1, were grown in the presence of different concentrations of ciprofloxacin (0, 62.5, 250, 1000 μg/mL) for 3 or 24 hours. The samples underwent PMA treatment, followed by DNA extraction, as described in Example 2. A primer set specific for K. pneumoniae that targets the gene phoE, derived from Shannon et al (doi:10.1016/j.scitotenv. 2007.02.039) was used in conjunction with the samples in a qPCR reaction.

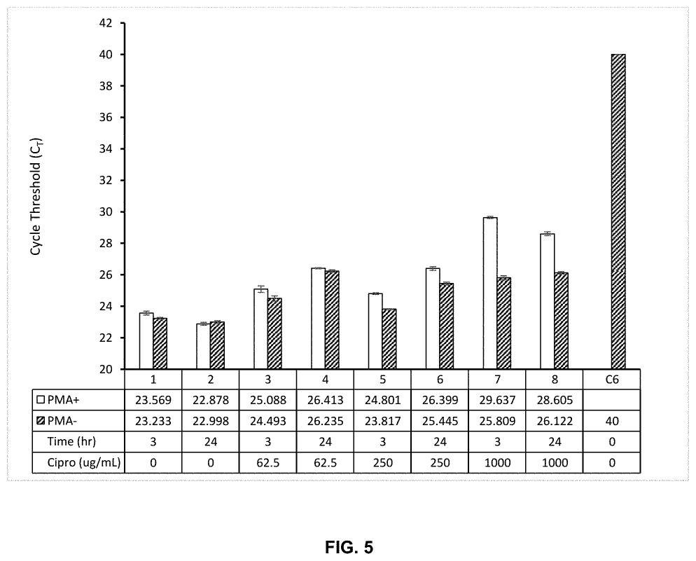

The results in FIG. 5 show that the PMA-treated sample for the highest ciprofloxacin concentration treatment (1000 μg/mL) in both the 3 and 24-hour treatments a have greater C_{T} (Cycle Threshold) value compared to their respective non-PMA-treated equivalent, suggesting that this PMA-qPCR can determine whether bacteria are susceptible to treatment with an antibiotic of interest.

### References

1. Thomas-White K, Brady M, Wolfe A J, et al: The Bladder Is Not Sterile: History and Current Discoveries on the Urinary Microbiome. Curr. Bladder Dysfunct. Rep. 2016; 11: 18-24.

2. Wolfe A J, Toh E, Shibata N, et al: Evidence of uncultivated bacteria in the adult female bladder. J. Clin. Microbiol. 2012; 50: 1376-1383. Available at: http://www.pubmedcentral.nih.gov/articlerender.fcgi?artid=3318548&tool=pmcentrez&rend ertype=abstract.

3. Siddiqui H, Lagesen K, Nederbragt A J, et al: Alterations of microbiota in urine from women with interstitial cystitis. BMC Microbiol. 2012; 12: 205. Available at: http://www.ncbi.nlm.nih.gov/pubmed/22974186.

4. Brubaker L and Wolfe A J: The new world of the urinary microbiota in women. Am. J. Obstet. Gynecol. 2015; 213: 644-649.

5. Hilt E E, McKinley K, Pearce M M, et al: Urine is not sterile: Use of enhanced urine culture techniques to detect resident bacterial flora in the adult female bladder. J. Clin. Microbiol. 2014; 52: 871-876. Available at: http://www.ncbi.nlm.nih.gov/pubmed/24371246.

6. Buc̆evié Popović V, S̆itum M, Chow C-E T, et al: The urinary microbiome associated with bladder cancer. Sci. Rep. 2018; 8: 12157. Available at: http://www.ncbi.nlm.nih.gov/pubmed/30108246.

7. Bajic P, Van Kuiken M E, Burge B K, et al: Male Bladder Microbiome Relates to Lower Urinary Tract Symptoms. Eur. Urol. Focus 2020; 6: 376-382.

8. Fouts D E, Pieper R, Szpakowski S, et al: Integrated next-generation sequencing of 16S rDNA and metaproteomics differentiate the healthy urine microbiome from asymptomatic bacteriuria in neuropathic bladder associated with spinal cord injury. J. Transl. Med. 2012; 10: 1. Available at: http://www.ncbi.nlm.nih.gov/pubmed/22929533.

9. Carini P, Marsden P J, Leff J W, et al: Relic DNA is abundant in soil and obscures estimates of soil microbial diversity. Nat. Microbiol. 2016; 2: 16242.

10. Lennon J T, Muscarella M E, Placella S A, et al: How, When, and Where Relic DNA Affects

Microbial Diversity. MBio 2018; 9.

11. Fittipaldi M, Rodriguez N J P, Codony F, et al: Discrimination of infectious bacteriophage T4 virus by propidium monoazide real-time PCR. J. Virol. Methods 2010; 168: 228-232.

12. Gobert G, Cotillard A, Fourmestraux C, et al: Droplet digital PCR improves absolute quantification of viable lactic acid bacteria in faecal samples. J. Microbiol. Methods 2018; 148: 64-73.

13. Lu J, Zheng H, Chu P, et al: Direct detection from clinical sputum samples to differentiate live and dead Mycobacterium Tuberculosis. J. Clin. Lab. Anal. 2019; 33: e22716.

14. Brauge T, Midelet-Bourdin G and Soumet C: Viability Detection of Foodborne Bacterial

Pathogens in Food Environment by PMA-qPCR and by Microscopic Observation. Methods Mol. Biol. 2019; 1918: 117-128.

15. Cattani F, Barth V C J, Nasário J S R, et al: Detection and quantification of viable Bacillus cereus group species in milk by propidium monoazide quantitative real-time PCR. J. Dairy Sci. 2016; 99: 2617-2624.

16. Hung C: A murine model of urinary tract infection. Nat. Protoc. 2009; 4: 1230-1243.

17. Taskin B, Gozen A G and Duran M: Selective quantification of viable Escherichia coli bacteria in biosolids by quantitative PCR with propidium monoazide modification. Appl. Environ. Microbiol. 2011; 77: 4329-4335.

18. Yang B and Bankir L: Urea and urine concentrating ability: new insights from studies in mice. Am. J. Physiol. Renal Physiol. 2005; 288: F881-96.

19. Guillard T, Cambau E, Chau F, et al: Ciprofloxacin treatment failure in a murine model of pyelonephritis due to an AAC(6′)-Ib-cr-producing Escherichia coli strain susceptible to ciprofloxacin in vitro. Antimicrob. Agents Chemother. 2013; 57: 5830-5835.

20. Chamberlain C A, Rubio V Y and Garrett T J: Impact of matrix effects and ionization efficiency in non-quantitative untargeted metabolomics. Metabolomics 2019; 15: 135.

21. Oliver J D: The viable but nonculturable state in bacteria. J. Microbiol. 2005; 43 Spec No: 93-100.

22. Coutard F, Pommepuy M, Loaec S, et al: mRNA detection by reverse transcription-PCR for monitoring viability and potential virulence in a pathogenic strain of Vibrio parahaemolyticus in viable but nonculturable state. J. Appl. Microbiol. 2005; 98: 951-961.

1. A method of detecting and/or quantitating metabolically active (e.g., live) microorganisms in a urine sample, the method comprising amplifying DNA in the urine sample pre-treated with a DNA-crosslinking agent that preferentially or exclusively penetrates metabolically inactive (e.g., dead) but not metabolically active (e.g., live) microorganisms, wherein the urine sample comprises, consists essentially of, or consists of insoluble components separated from the aqueous portion of urine; and wherein the microorganism comprises bacteria, yeast, virus, or combinations thereof.

2. The method of claim 1, wherein the urine sample is obtained by re-suspending said insoluble components in a buffer after centrifuging the urine to precipitate insoluble components.

3. The method of claim 2, wherein the buffer is compatible for DNA amplification, such as PBS.

4. The method of claim 1, wherein the insoluble components are precipitated from the urine by centrifugation at about 5,000 g or above, for at least about 8 minutes.

5. The method of claim 2, wherein the urine is diluted by the buffer (e.g., 1:2 dilution) before centrifugation.

6. The method of claim 1, wherein the DNA-crosslinking agent is propidium monoazide (PMA), propidium monoazide derivatives such as PMaxx and PMaxx Plus, ethidium monoazide bromide (EMA), a chromium metabolite (thiol reactions), a simple aryl azide, a fluorinated aryl azide such as a nitrene generating reagent, or a benzophenone derivative.

7. The method of claim 6, wherein the DNA-crosslinking agent is PMA.

8. The method of claim 1, wherein the urine sample is pre-treated with the DNA-crosslinking agent (e.g., 200 μM PMA) by incubating in dark for 15 or more minutes, followed by LED-light-induced crosslinking for 20 or more minutes.

9. The method of claim 1, wherein DNA amplification is performed by polymerase chain reaction (PCR), rolling circle amplification (RCA), loop mediated isothermal amplification (LAMP), nucleic acid sequence based amplification (NASBA), strand displacement amplification (SDA), multiple displacement amplification (MDA), ligase chain reaction (LCR), helicase-dependent amplification (HDA), or ramification amplification method (RAM).

10. The method of claim 9, wherein DNA amplification is performed by quantitative PCR (qPCR).

11. The method of claim 9, wherein the PCR simultaneously amplify two or more marker genes from the bacteria (e.g., comprising the uidA gene of E coli.).

12. The method of claim 9, wherein the PCR amplifies an antibiotic resistance gene from the bacteria, and/or a bacterial species-specific gene.

13. The method of claim 1, wherein the bacteria comprise E. coli, Pseudomonas spp. (such as Pseudomonas aeruginosa), Klebsiella spp. (such as Klebsiella pneumoniae), Proteus spp. (such as Proteus mirabilis), Enterococcus spp. (such as Enterococcus faecalis), Enterobacter, Coagulase-negative Staphylococci, Staphylococcus aureus, and/or Acinobacter; and/or wherein the microorganism comprises Acinetobacter baumannii, Actinotignum schaalii, Aerococcus urinae, Alloscardovia omnicolens, Candida albicans, Candida auris, Candida glabrata, Candida parapsilosis, Citrobacter freundii, Citrobacter koseri, Corynebacterium riegelii, Enterococcus faecalis, Enterococcus faecium, Escherichia coli, Klebsiella oxytoca, Coagulase-neg. staphylococci, Viridans group streptococci, Enterobacter group, BK virus, HHV-5 (CMV), HHV-6, HHV-1, HHV-2 (HSV 1/2), JC virus, Klebsiella pneumoniae, Morganella morganii, Mycobacterium tuberculosis, Mycoplasma hominis, Pantoea agglomerans, Proteus mirabilis, Providencia stuartii, Pseudomonas aeruginosa, Serratia marcescens, Staphylococcus aureus, Streptococcus agalactiae, Ureaplasma urealyticum, Chlamydia trachomatis, Neisseria gonorrhoeae, Trichomonas vaginalis, or combinations thereof.

14. The method of claim 1, wherein said urine is from a mammalian subject (e.g., human or mouse).

15. The method of claim 14, wherein the human has, suspected to have, is predisposed to or is at high risk of having UTI (uninary tract infection).

16. The method of claim 15, wherein the UTI is cystitis, pyelonephritis, or complicated UTI (cUTI).

17. The method of claim 14, wherein the mammalian subject is a human having urinary catheterization (e.g., human having chronic catheter) or have had urinary catheterization (e.g., human having intermittent catheterization).

18. The method of claim 14, wherein the mammalian subject is a human undergoing or having completed treatment (e.g., antibiotic treatment) for UTI.

19. The method of claim 14, wherein the mammalian subject is a human at increased risk for recurrent UTI, such as human with intermittent catheterization, Spina bifida, lower urinary tract dysfunction, diabetes, and/or immunosuppression.

20. The method of claim 14, wherein the mammalian subject is a female human, such as a woman who is pregnant, or a woman who has given recent (e.g., within 1, 2, 3 day, 1, 2, 3, weeks, 1, 2, 3 months) vaginal birth.

21. The method of claim 14, wherein the mammalian subject is a geriatrics patient.
